(19) United States
(12) Patent Application Publication
(10) Pub. No.: US 2017/0189445 A1
(43) Pub. Date: Jul. 6, 2017

(54) XENON ASSOCIATED WITH AN NMDA RECEPTOR ANTAGONIST FOR CONTROLLING TUMOR PROLIFERATION IN THE CENTRAL NERVOUS SYSTEM

(71) Applicants: L'Air Liquide, Société Anonyme pour I'Etude et I'Exploitation des Procédés Georges Claude, Paris (FR); L'Institut du Cerveau et de la Moelle Epiniere, Paris (FR)

(72) Inventors: Patrick MICHEL, Paris (FR); Jérémie LAVAUR, Paris (FR); Etienne HIRSCH, Versailles (FR); Marc LEMAIRE, Paris (FR)

(21) Appl. No.: 15/316,227

(22) PCT Filed: Jun. 8, 2015
(86) PCT No.: PCT/FR2015/051515

§ 371 Date: Dec. 5, 2016

(30) Foreign Application Priority Data
Jun. 20, 2014 (FR) 1455709

(51) Int. Cl. A61K 33/00; A61K 31/13; A61K 9/00; A61K 45/06

(52) U.S. Cl. CPC A61K 33/00; A61K 45/06; A61K 31/13; A61K 9/007

(57) ABSTRACT

The invention relates to a drug combination including gaseous xenon and at least one NMDA receptor antagonist in liquid or solid form. In order to treat or slow tumour proliferation of cells in the contral nervous system in a human being, in particular astrocyte glia and/or the percursors thereof. The NMDA receptor antagonist is perferably memantine or altromemantine. The proportion of xenonis 10% to 80% by volume.

### CROSS REFERENCE TO RELATED APPLICATIONS

This application is a 371 of International PCT Application PCT/FR2015/051515, filed Jun. 8, 2015, which claims priority to French Patent Application No. 1455709, filed Jun. 20, 2014, the entire contents of which are incorporated herein by reference.

### BACKGROUND

The invention relates to the use of xenon gas as an inhalable drug used in combination with an N-methyl-D-aspartate (NMDA) glutamate receptor antagonist, in particular memantine or nitromemantine, for treating or slowing down tumor proliferation of the central nervous system, in particular of glial cells of astrocyte type and/or of precursors thereof.

NMDA receptors/channels are molecular entities of the plasma membrane of neuronal cells. These receptors are the target of glutamate molecules released into the synaptic and extrasynaptic space, glutamate being an excitatory neurotransmitter which provides communication from one nerve cell to another.

NMDA receptors/channels are also present on glial cells which are non-neuronal cells of the central nervous system (Lee et al., PloSOne, 2010; Dzamba et al., Curr Neuropharmacol, 2013).

The stimulation of NMDA receptors on glial cells of astrocyte type contributes to the control of glutamate release by axonal nerve endings and to the modulation of brain plasticity (Conti et al., Cereb. Cortex, 1999).

This function is however disrupted in tumors of glial cells of astrocyte type and/or of precursors thereof (Takano et al., Nat Med, 2001; Rzeski et al., Biochem Pharmacol, 2002). Indeed, it has been observed that blockage of NMDA glutamate receptors by various antagonists of these receptors, in particular memantine, substantially reduces the proliferation of glial tumor cells of astrocyte type and/or of precursors thereof, via a mechanism which requires a reduction in calcium flux in these cells (Rzeski et al., Proc. Natl. Acad. Sc. USA, 2001).

However, the effect of memantine is limited because this molecule is not without adverse effects, such as confusion, dizziness, sleepiness, headaches, insomnia, agitation, hallucinations, vomiting, anxiety, etc.

The problem is therefore that of providing a treatment which makes it possible to slow down or minimize any tumor proliferation of the cells of the central nervous system, in particular of glial cells of astrocyte type and/or of precursors thereof, while limiting the doses of memantine used and therefore the adverse effects associated with the use of this compound, so as to preserve the integrity and the functions of the nervous tissue in a human being, i.e. a human patient.

### SUMMARY

The solution according to the present invention relates to a drug combination comprising xenon gas and at least one NMDA receptor antagonist in liquid or solid form for treating or slowing down tumor proliferation of the central nervous system, in particular of glial cells, in particular of glial cells of astrocyte type and/or of precursors thereof, in a human being, in particular an NMDA receptor antagonist of memantine type or a memantine-derived compound, such as nitromemantine.

In other words, the present invention relates to a drug containing xenon gas, for use by inhalation in combination with at least one NMDA receptor antagonist in liquid or solid form, for treating or slowing down tumor proliferation of cells of the central nervous system, in particular of glial cells, preferably of astrocyte type and/or of precursors thereof.

Indeed, in the context of the present invention, it has been demonstrated that the combination of xenon and NMDA receptor antagonist, such as memantine or a memantine-derived compound, in particular nitromemantine, results in a synergistic action of these compounds and that such a combination can constitute a promising treatment for combating tumor proliferation of cells of the central nervous system, in particular of glial cells of astrocyte type and/or of precursors thereof.

Such a combination is based in particular on the modes of action of these compounds.

Thus, xenon has excitatory glutamatergic signaling pathway-inhibiting properties via its antagonistic action on NMDA receptors (Dinse et al., Br J Anaesth, 2005).

Consequently, the xenon/memantine or xenon/nitromemantine combination results in a synergy of action at the level of glutamatergic receptors, in particular of NMDA type, without risking increasing the adverse effects of memantine.

In other words, xenon makes it possible to increase the beneficial effects of memantine or of nitromemantine by a synergistic effect, but without causing the adverse effects of memantine or of nitromemantine.

As appropriate, the gaseous drug according to the invention can meet one or more of the following criteria:
the NMDA receptor antagonist is in solid form, in particular in tablet or gel capsule form;
the NMDA receptor antagonist is memantine;
the NMDA receptor antagonist is a memantine-derived compound, for example nitromemantine;
the NMDA receptor antagonist is nitromemantine;
the tumor proliferation relates to cells of the central nervous system;
the tumor proliferation relates to glial cells;
the tumor proliferation relates to glial cells of astrocyte type and/or precursors thereof;
the daily dose of NMDA receptor antagonist administered to the patient is less than or equal to 20 mg/day;
the xenon gas is administered to the patient before, concomitantly with or after administration of the NMDA receptor antagonist, preferably after administration of the NMDA receptor antagonist;
the xenon is contained in a gas mixture additionally containing oxygen, preferably at least 21% by volume of oxygen;
the gas mixture consists of xenon and oxygen;
the gas mixture consists of xenon, nitrogen and oxygen;
the patient is a human being;
the patient to be treated is a man or a woman;
the xenon is in an effective amount;
the amount of xenon is non-anesthetic, i.e. sub-anesthetic;
it contains between 10% and 80% by volume of xenon;
it contains between 15% and 80% by volume of xenon;
it contains at least 20% by volume of xenon;
it contains less than 60% by volume of xenon;
it contains 75% by volume of xenon;
it contains between 20% and 50% by volume of xenon;
it contains between 20% and 40% by volume of xenon;
the xenon is mixed with oxygen just before or at the time of its inhalation by the patient or is in the form of a “ready-to-use” gas mixture in a pre-mix with oxygen, and optionally contains another gas compound, for example nitrogen;
the gas containing the xenon is administered to the patient by inhalation;
the gas containing the xenon is administered to the patient one or more times per day;
the gas containing the xenon is administered to the patient for an inhalation time of a few minutes to a few hours, typically between 15 minutes and 6 hours, preferentially less than 4 hours;
the duration, the dosage regimen and the frequency of administration depend on the progression of the neurological condition of the patient under consideration and will preferentially be set by the physician or care staff according to the neurological condition of the patient under consideration;
the xenon is packaged in a gas cylinder having a volume (water equivalent) ranging up to 50 liters, typically of about from 0.5 a 15 liters and/or at a pressure of less than or equal to 350 bar absolute, typically a pressure of between 2 and 300 bar;
the xenon is packaged in a gas bottle equipped with a valve or a pressure regulator that is integrated, making it possible to control the flow rate and optionally the pressure of the gas delivered;
the xenon gas is packaged in a gas cylinder made of steel, aluminum or composite materials;
during the treatment, the xenon gas is administered to the patient by inhalation by means of a face mask or nasal mask or of nasal goggles or by means of any other system for administration of an inhalable gas.

According to another aspect, the invention also relates to a method of therapeutic treatment for treating or slowing down tumor proliferation of the cells of the central nervous system, in particular of glial cells, preferably of glial cells of astrocyte type and/or of precursors thereof, in a human patient, in which method:
i) a human patient suffering from tumor proliferation of the cells of the central nervous system, in particular of glial cells of astrocyte type, and/or of precursors thereof, is identified,
ii) a gaseous drug containing xenon is administered to said patient by inhalation, and
iii) at least one NMDA receptor antagonist in liquid or solid form is administered to said patient,
   so as to make it possible to inhibit or slow down the tumor proliferation in said patient.

Preferably, in step i):
the human patient is a child, a man or a woman;
the patient is identified by a physician or the like;
the proliferation is identified by imaging, biological tests, biopsies, etc.;
the tumor proliferation of the cells of the central nervous system is observed in particular at the level of the glial cells of astrocyte type and/or of precursors thereof;
the tumor proliferation of the cells of the central nervous system results from an excessive entry of Ca²⁺ ions into cells of astrocyte type.

Preferably, in step ii):
at least one NMDA receptor antagonist in solid form is administered to said patient;
at least one NMDA receptor antagonist is administered, preferably enterally, i.e. orally;
at least one NMDA receptor antagonist in tablet or gel capsule form is administered to said patient;
memantine, or a memantine derivative or compound is administered to the patient as NMDA receptor antagonist;
memantine or nitromemantine is administered to the patient as NMDA receptor antagonist;
a daily dose of NMDA receptor antagonist of less than or equal to 20 mg/day is administered to the patient;
at least one NMDA receptor antagonist is administered to said patient before, concomitantly with, or after inhalation of xenon by the patient.

Preferably, in step iii):
the duration, the dosage regimen and the frequency of administration of the xenon are chosen and/or set according to the progression of the neurological condition of the patient under consideration;
an effective amount of xenon is administered;
a non-anesthetic amount of xenon is administered;
from 10% to 75% by volume of xenon, preferably between 20% and 50% by volume of xenon, is administered;
the xenon is mixed with oxygen before or at the time of its inhalation by the patient, preferably with at least 21% by volume of oxygen;
a ready-to-use gas mixture consisting of xenon and oxygen (binary mixture) or of xenon, oxygen and nitrogen (ternary mixture) is administered;
the xenon gas is administered to the patient one or more times per day;
the xenon gas is administered to the patient for an inhalation time of a few minutes to a few hours, typically between 15 minutes and 6 hours, preferentially less than 4 hours;
the xenon gas is administered by means of a face mask or nasal mask or of nasal goggles or by means of any other system or device for administration of gas to a patient.

### BRIEF DESCRIPTION OF THE DRAWINGS

The invention will now be understood more clearly by virtue of the following examples and with reference to the appended figures, given by way of illustration but without limitation, among which:

FIG. 1 illustrates the efficacy of a combination of xenon and an NMDA receptor antagonist in a cell model mimicking cell proliferation of glial cells of astrocyte type, and/or of precursors thereof;

FIG. 2 illustrates the efficacy of a combination of xenon and an NMDA receptor antagonist in a neuron-glia mixed culture cell model.

### DESCRIPTION OF PREFERRED EMBODIMENTS

### EXAMPLES

### Example 1

In order to demonstrate the efficacy of the combination of xenon and an NMDA receptor antagonist according to the present invention, a cell model of cultures enriched with cells of astrocyte type was set up, in which model the proliferation is amplified and maintained by the presence of mitogens, contained in particular in the fetal calf serum (FCS) that is used to supplement the culture medium.

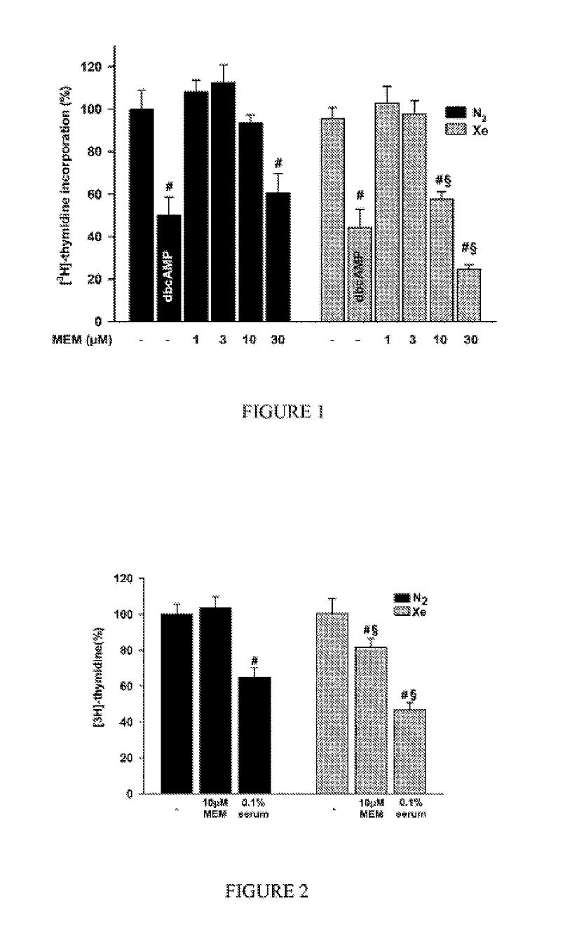

The technique set up is described hereinafter and the results obtained are reported in table 1 and illustrated in FIG. 1.

These results show the synergistic anti-proliferative effects of xenon and memantine in a cell model mimicking proliferation of glial cells of astrocyte type and/or of precursors thereof.

### Protocol for Obtaining Astrocyte Primary Cultures

Cultures are prepared from total brain of embryos, taken from gestating Wistar female rats on day 15.5 of gestation.

The method for producing the cultures enriched with astrocytes (cells immunopositive for glial fibrillary acidic protein or GFAP) comprises obtaining a homogeneous cell suspension of the entire embryonic anterior brain by mechanical dissociation, i.e. non-enzymatic dissociation, using Leibovitz L15 medium (Sigma Aldrich).

An aliquot of cell suspension corresponding to the brain tissue from 5 embryos is added to each 25 cm² flask (Costar) containing 5 ml of a feeder medium which will make it possible to stimulate the proliferation of the astrocytes and of precursors thereof (Sergent-Tanguy et al., J Neurosci Res, 2006), while at the same time eliminating the neuronal cells; the neurons do not adhere to the bottom of the flask in the absence of coating, unlike the astrocytes. The medium used is Minimum Essential Medium (MEM), containing 10% of fetal calf serum (FCS, Biowest) and 10% of horse serum (HS, Sigma Aldrich), 1 g/l of glucose, 2 mM of L-glutamine, 1 mM of sodium pyruvate, non-essential amino acids, and a penicillin/streptomycin cocktail. When confluence is reached (i.e. after 7-10 days of culture), the adherent cells (>90% of astrocytes) are placed in PBS, then detached by tapping the flasks. The cells recovered are centrifuged and mechanically triturated before being placed in culture. The astrocytes are then cultured in 48-well multiwell plates (Nunc). Approximately 30 000 cells are applied per culture well. The cells are placed in the presence of the same medium as previously, but with a serum supplementation, reduced to 2%.

No change of culture medium is carried out throughout the culture period.

### Pharmacological Treatments of the Cultures

The proliferation is triggered and maintained by the presence of FCS in the culture medium.

The NMDA receptor blocker, memantine (RD Systems), is added to the cultures just before exposure to the xenon. Dibutyryl cyclic AMP (dbcAMP), a lipophilic form of cAMP used as a reference anti-proliferative molecule (Mourlevat et al., Mol Pharmacol, 2003), is added at the same time as the memantine.

### Maintenance of the Cultures Under a Controlled Gaseous Atmosphere

Once the pharmacological treatments have been carried out, the multiwell dish plates containing the cells in culture and the plate used to humidify the internal compartment of the chamber are placed on a metal base which receives the Plexiglas incubation chamber. The two parts (base and Plexiglas chamber) are butt-joined together by screwing.

A gas mixture of interest comprising (% by volume): 20% of O₂, 5% of CO₂ and 75% of the gas tested is then injected into the incubation chamber, with open inlet and outlet valves, while at the same time controlling the output flow rate by means of a flow meter. The gases tested are nitrogen and xenon.

The reference output flow rate, set for air at 10 liters/min, is corrected according to the density of the mixture used.

When the CO₂ measurement reaches 5% at the outlet, the injection of the gas mixture is stopped and the chamber is made totally airtight by closing the inlet and outlet valves.

The exposure chamber is then placed in an enclosure at 37° C. throughout the experimental protocol.

### Measurement of the Cell Proliferation by Tritiated Thymidine Accumulation

After the airtightness has been broken by opening the inlet and outlet valves and unscrewing the chamber from its base, the cultures are placed in the presence of PBS containing 5 mM of glucose and incubated for 2 h with tritiated thymidine (Perkin, 70-90 Ci/mmol; 0.5 μCi/well) in order to analyze the cell proliferation according to a method previously described by Rousseau and colleagues (Mol Pharmacol, 2013).

The results obtained in the cell proliferation model in the presence of FCS reveal synergistic inhibitory effects between the xenon and the memantine which are summarized in the following table 1 and represented in FIG. 1.

TABLE 1
Summary of the principal results of the in vitro study on astrocyte-
enriched cultures
Cultures enriched with astrocytes on D3

| | Gas mixture | |
|---|---|---|
| | (20% O₂ + 5% CO₂ + | Reduction |
| | 75% gas tested); % by | in |
| Treatments D0-D3 (3 days) | volume | proliferation |
| Control group I | N₂ | − |
| Control group II | Xe | − |
| dbcAMP (1 mM) | N₂ | ++ |
| dbcAMP (1 mM) | Xe | ++ |
| Memantine (1 μM) | N₂ | − |
| Memantine (1 μM) | Xe | − |
| Memantine (3 μM) | N₂ | − |
| Memantine (3 μM) | Xe | − |
| Memantine (10 μM) | N₂ | − |
| Memantine (10 μM) | Xe | ++ |
| Memantine (30 μM) | N₂ | ++ |
| Memantine (30 μM) | Xe | +++ |

In the above table, a favorable response, synonymous with inhibition of the proliferation of glial cells of astrocyte type in the presence of the treatments of interest, is denoted by a “+”, “++”, “+++” or “++++” (++++=absence of proliferation) sign. Conversely, an absence of response is represented by a “−” sign, synonymous with non-efficacy with respect to cell proliferation.

Memantine acts by blocking NMDA receptors and xenon acts by blocking these same receptors but probably via a distinct mechanism, involving blocking of the binding site for glycine, an amino acid which acts as a coactivator of the receptor.

FCS induces and maintains the cell proliferation process due to its high content of mitogenic factors.

In the light of the results illustrated in FIG. 1, it is noted that the combination of xenon and an NMDA receptor antagonist, namely in this case memantine, produces a synergistic anti-proliferative effect greater than that produced by each treatment individually. In fact, an actual synergy of action of the xenon/memantine combination is set up with respect to the proliferation mechanism.

First of all, these tests show that the proliferation in the presence of serum mitogens is sustained under an atmosphere containing 75 vol % of nitrogen. This proliferation can be significantly reduced by a lipophilic analog of cAMP, dibutryl-cAMP.

When xenon replaces nitrogen, no significant anti-proliferative effect is observed.

Memantine under a nitrogen-enriched atmosphere also has no anti-proliferative effect up to 10 μM. However, treatment with 10 μM of memantine in a xenon-enriched atmosphere results in a significant anti-proliferative effect (−42.5%).

Memantine at 30 pM exerts an anti-proliferative effect on its own (−39.3%) in a nitrogen-enriched atmosphere and this effect is strongly potentiated under a xenon-enriched atmosphere (−75.2%).

The results represented diagrammatically in FIG. 1 illustrate the synergistic protective effects of xenon and memantine in a cell model mimicking proliferation of glial cells of astrocyte type and/or of precursors thereof.

These results were obtained on cultures enriched with glial cells of astrocyte type, obtained from rat brain. These cultures were maintained, as soon as they were placed in culture and up to D3 in vitro, in an atmosphere containing 75% of nitrogen (N₂ 75) or 75% of xenon (Xe 75), in the presence or absence of memantine (MEM), tested at 1, 3, 10 or 30 μM or in the presence of a soluble analog of cAMP, dibutyryl-cAMP, used as reference anti-proliferative molecule.

At the end of the treatments, the cultures are recovered for a cell proliferation measurement, using tritiated thymidine incorporation as evaluation index.

The results are expressed in % (±SEM, n=6) of the mean values of the cultures maintained under 75% of nitrogen (control condition).

Thus, the statistical study carried out by a one-way analysis of variance (ANOVA) followed by a Student-Newman-Keuls Method test for multiple paired comparisons (n=6 for each experimental point), demonstrates that:
in an atmosphere containing 75% of nitrogen, the astrocyte proliferation is reduced in the presence of 30 μM of memantine or of 1 mM of dbcAMP (# p<0.05, vs control cultures maintained under 75% of nitrogen);
in an atmosphere containing 75% of xenon, the astrocyte proliferation is reduced from as early as 10 μM of memantine with 1 mM of dbcAMP (# p<0.05, vs control cultures maintained under 75% of nitrogen);
the anti-proliferative potential of memantine is either revealed or increased under a xenon-enriched atmosphere (§ p<0.05, vs cultures exposed to the same concentrations of memantine under 75% of nitrogen).

Therefore, xenon, when it is combined with memantine or with nitromemantine acting as NMDA receptor antagonist, produces a synergistic effect in the treatment or slowing down of central nervous system tumor proliferations of glial cells of astrocyte type and/or of precursors thereof.

### Example 2

In order to confirm that the synergistic effect of the combination of xenon and NMDA receptor antagonist according to the present invention is real, another neuron-glia mixed culture cell model was set up, in which model the proliferation of the glial cells of astrocyte type and/or of precursors thereof is observed in the presence of neuronal cells (Rousseau et al., Mol Pharmacol, 2013).

The technique used is described below and the results obtained are summarized in table 2 and illustrated in FIG. 2 which shows the synergistic inhibitory effects of xenon and memantine in a cell model mimicking proliferation of glial cells of astrocyte type and/or of precursors thereof in the presence of neuronal cells.

### Protocol for Obtaining the Neuron-Glia Mixed Cultures

Cultures are prepared from mesencephalon of rat embryos taken from female Wistar rats, on day 15.5 of gestation.

The use of the mesencephalon cultures comprises obtaining a homogeneous cell suspension by mechanical dissociation, i.e. non-enzymatic dissociation, of the embryonic tissue, using Leibovitz L15 medium (Sigma Aldrich).

Aliquots of this suspension are added to Nunc 48-well multiwell plates, which have been precoated with a thin layer of polyethyleneimine (1 mg/ml, borate buffer, pH 8.3) so as to enable adhesion of the neuronal cells (cf. Toulorge et al., Faseb J, 2011).

The seeding density is between approximately 80 000 and 100 000 cells/culture well.

The mesencephalon cultures are maintained in MEM culture medium, containing 1 g/l of glucose, 2 mM of L-glutamine, 1 mM of sodium pyruvate, non-essential amino acids and a penicillin/streptomycin cocktail. During the first week of culture, this medium also contains 10% of fetal calf serum (FCS, Biowest) and 10% of horse serum (HS, Sigma Aldrich), but from the second week, the concentrations of the two sera are reduced (Gao et al., J Neurosci, 2002).

Up to the time when the effects of the gases of interest are evaluated, the cultures are placed in a conventional enclosure thermostatically controlled at 37° C., in which the CO₂ is maintained at 5% by volume and wherein the atmosphere is saturated with water.

The glial proliferation process occurs as soon as the cells are placed in culture. It is analyzed during the second week in vitro when the serum supplementation is reduced to 2% or in some cases to 0.1%.

### Pharmacological Treatments of the Cultures

After reduction of the FCS and HS concentrations on D7, the NMDA receptor blocker, memantine, is added to the cultures before placing them under a controlled gaseous atmosphere.

Maintenance of the Cultures under a Controlled Gaseous Atmosphere

Once the pharmacological treatments have been carried out, the multiwell plates containing the cells in culture and the plate used for humidifying the internal compartment of the chamber are placed on a metal base which receives the Plexiglas incubation chamber. The two parts (base and Plexiglas chamber) are butt-joined together by screwing.

A gas mixture of interest comprising (% by volume): 20% of O₂, 5% of CO₂ and 75% of gas tested is then injected into the incubation chamber, with open inlet and outlet valves, while at the same time controlling the output flow rate by means of a flow meter. The gases tested are nitrogen and xenon.

The reference output flow rate, set for air at 10 liters/min, is corrected according to the density of the mixture used.

When the CO₂ measurement reaches 5% at the outlet, the injection of the gas mixture is stopped and the chamber is made totally airtight by closing the inlet and outlet valves.

The exposure chamber is then placed in an enclosure at 37° C. for the 7 days of the experimental protocol.

### Measurement of the Cell Proliferation by Thymidine Incorporation

On D14, after the airtightness has been broken by opening the inlet and outlet valves and unscrewing the incubation chamber from its base, the cultures are subjected to the tritiated-thymidine proliferation test according to the protocol described in example 1, using as a basis the technique described by Rousseau and colleagues (Mol. Pharmacol., 2013).

The results obtained in the astrocyte cell proliferation model reveal synergistic inhibitory effects between xenon and memantine which are summarized in the following table 2 and represented in FIG. 2. It is also noted that reducing the concentrations of the two sera to 0.1% strongly reduces the astrocyte proliferation, thereby demonstrating the key role of the serum mitogens in the proliferative process.

TABLE 2
Summary of the principal results of the in vitro
study in the neuron-glia mixed culture model
D14 neuron-glia mixed cultures

| | Gas mixture | |
|---|---|---|
| | (20% O₂ + 5% CO₂ + | |
| | 75% gas tested); | Inhibition of |
| Treatments D7-D14 (7 days) | % by volume | proliferation |
| Control group, sera at 2% | N₂ | − |
| Xe group, sera at 2% | Xe | − |
| Memantine (10 μM) group, | N₂ | − |
| sera at 2% | | |
| Xe + memantine (10 μM) | Xe | + |
| group, sera at 2% | | |
| Group, sera at 0.1% | N₂ | ++ |
| Xe group, sera at 0.1% | Xe | +++ |

In the light of the results represented in FIG. 2, it is noted that the combination of xenon and an NMDA receptor antagonist, namely in this case memantine, produces a synergistic inhibitory effect with respect to the astrocyte proliferation. The treatment of the cultures with memantine applied under nitrogen or the maintenance of the cultures in a xenon-enriched atmosphere have no impact on the proliferation. The decreasing of the serum concentrations in the culture medium also results in a reduction in the astrocyte proliferation, thus demonstrating that the memantine/xenon combination is active against a mechanism activated by serum mitogens. In fact, an actual synergy of action of the xenon/memantine combination is set up.

The results described in FIG. 2 demonstrate the synergistic inhibitory effects of xenon and memantine in the neuron-glia mixed culture cell model mimicking proliferation of glial cells of astrocyte type and/or of precursors thereof.

These results were obtained on rat mesencephalon cultures which were placed, starting from day 7 of culture, in a medium supplemented with 2% of FCS and of HS under an atmosphere containing 75% of nitrogen (N₂ 75) or 75% of xenon (Xe 75), in the presence or absence of memantine (MEM), tested at 10 μM. Some experiments were also carried out by reducing the concentrations of the 2 types of serum to 0.1%, while maintaining the cells under a nitrogen-enriched or xenon-enriched atmosphere.

The cultures are then recovered for a cell proliferation measurement using tritiated thymidine.

The results are expressed in % (±SEM) of the cultures on D14 maintained under nitrogen (control condition).

Thus, the statistical study carried out by means of an ANOVA (ANalysis Of VAriance), followed by a Fisher test (n=13-15 for each experimental point) demonstrates that:
in an atmosphere containing 75% of nitrogen, the astrocyte proliferation is decreased only if the serum concentrations are reduced from 2% to 0.1% (# p<0.05, vs control cultures maintained under 75% of nitrogen);
in an atmosphere containing 75% of xenon, the astrocyte proliferation is decreased if the serum concentrations are reduced to 0.1% or if the cultures are treated with memantine (# p<0.05, vs control cultures maintained under 75% of nitrogen);
xenon reveals the anti-proliferative effects of a treatment with memantine or improves those produced by reducing the serum concentrations (§ p<0.05, vs corresponding cultures exposed for the same treatments under 75% of nitrogen).

Therefore, xenon, when it is combined with an NMDA receptor antagonist, such as memantine or nitromemantine acting as an NMDA receptor antagonist, produces a synergistic effect in the treatment or slowing down of central nervous system tumor proliferations of glial cells of astrocyte type and/or of precursors thereof.

In other words, according to the invention, xenon gas can be used to produce an inhalable drug which makes it possible to treat or slow down, when it used in combination with at least one NMDA receptor antagonist in liquid or solid form, tumor proliferation of cells in the central nervous system in a human being.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims. The present invention may suitably comprise, consist or consist essentially of the elements disclosed and may be practiced in the absence of an element not disclosed. Furthermore, if there is language referring to order, such as first and second, it should be understood in an exemplary sense and not in a limiting sense. For example, it can be recognized by those skilled in the art that certain steps can be combined into a single step.

The singular forms “a”, “an” and “the” include plural referents, unless the context clearly dictates otherwise.

“Comprising” in a claim is an open transitional term which means the subsequently identified claim elements are a nonexclusive listing (i.e., anything else may be additionally included and remain within the scope of “comprising”). “Comprising” as used herein may be replaced by the more limited transitional terms “consisting essentially of” and “consisting of” unless otherwise indicated herein. “Providing” in a claim is defined to mean furnishing, supplying, making available, or preparing something. The step may be performed by any actor in the absence of express language in the claim to the contrary.

Optional or optionally means that the subsequently described event or circumstances may or may not occur. The description includes instances where the event or circumstance occurs and instances where it does not occur.

Ranges may be expressed herein as from about one particular value, and/or to about another particular value. When such a range is expressed, it is to be understood that another embodiment is from the one particular value and/or to the other particular value, along with all combinations within said range.

All references identified herein are each hereby incorporated by reference into this application in their entireties, as well as for the specific information for which each is cited.

1. A drug combination comprising a xenon containing gas and at least one NMDA receptor antagonist in liquid or solid form, for treating or slowing down tumor proliferation of cells in the central nervous system in a human being.

2. The drug combination of claim 1 wherein the xenon containing gas contains between 10% and 80% by volume of xenon gas.

3. The drug combination of claim 1 wherein the at least one NMDA receptor antagonist is in solid form.

4. The drug combination of claim 1 wherein the cells are glial cells.

5. The drug combination of claim 4 wherein the glial cells are of the astrocyte cell type and/or of precursors thereof.

6. The drug combination as of claim 1 wherein the NMDA receptor antagonist is memantine or nitromemantine.

7. The drug combination of claim 1 wherein the xenon containing gas additionally contains at least 21% by volume of oxygen.

8. The drug combination of claim 1 wherein the xenon containing gas a mixture consisting of either xenon and oxygen or a mixture consisting of xenon, oxygen and nitrogen.

9. The drug combination of claim 2 wherein the xenon containing gas contains at least 20% by volume of xenon gas.

10. The drug combination of claim 2 wherein the xenon containing gas contains less than 75% by volume of xenon gas.

11. A drug therapy for treating or slowing down tumor proliferation of cells in the central nervous system in a human being comprising administering to the human being a xenon gas and at least one NMDA receptor antagonist in liquid or solid form to thereby treat or slow down proliferation of cells in the central nervous system in a human being.

12. The method of claim 11 wherein the xenon gas is 20%-60% by volume of a gaseous composition administered to the human being by inhalation.

13. The method of claim 12, wherein the gaseous composition further contains at least 21% oxygen gas.

14. The method of claim 12, wherein the at least one NMDA receptor antagonist in liquid or solid form is memantine or nitromemantine.

15. The method of claim 12, wherein the cells are glial cells of the astrocyte cell type and/or of precursors thereof.
